(19) United States
(12) Patent Application Publication
(10) Pub. No.: US 2010/0191310 A1
(43) Pub. Date: Jul. 29, 2010

(54) Communication-Anchor Loop For Injectable Device

(75) Inventor: Mark J. Bly, Falcon Heights, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Corventis, Inc., San Jose, CA (US)

(21) Appl. No.: 12/509,678

(22) Filed: Jul. 27, 2009

Related U.S. Application Data
(60) Provisional application No. 61/084,567, filed on Jul. 29, 2008.

(51) Int. Cl. A61N 1/08

(52) U.S. Cl. 607/60

(57) ABSTRACT

An injectable electronics device has a housing sized to fit within an injection tool lumen with one or more electrical components position within the housing, and a self-expanding loop antenna coupled to at least one electrical component within the housing. The self-expanding loop antenna is expandable from a first compressed shape to a second expanded shape.

### CROSS REFERENCE TO RELATED APPLICATION DATA

The present application claims the benefit under 35 USC 119(e) of U.S. Provisional Application No. 61/084,567 filed Jul. 29, 2008; the full disclosure of which is incorporated herein by reference in its entirety.

### BACKGROUND OF THE INVENTION

1. Field of the Invention

The present invention relates to an injectable electronic device having an attached compressible loop. The compressible loop is coupled to electronics within the device and serves as an antenna for communication and/or energy transfer. The compressible loop can also aid in anchoring the device at a desired site within a patient.

In at least some instances, an electronic device positioned within a patient's body to measure patient data and communicate outside the body can be somewhat invasive and larger than would be ideal in at least some instances. The electronic device may have some type of antenna structure coupled to electronics within a housing of the device. For example, the antenna structure may be an inductive coil loop positioned with the electronics within the housing. This use of the loop inside the housing can allow all the components to be contained within the housing to maintain hermetic sealing of the device, and the housing may allow signals to pass there through, such that the loop can be used for charging and communication. However, in at least some instances this use of the loop inside the hermetically sealed housing can limit the housing material to non-metallic materials, such as glass, ceramic, polymers, etc. The non-metallic housings can result in increased wall thickness to maintain hermetic sealing and structural stability, such that the size and invasiveness of the device can increase in at least some instances.

With the current state of the art, the presence of the inductive coil loop can result in packaging and sizing that is less than ideal for an injectable device in at least some instances. Although a larger coil/loop, may use less energy for charging and communication, the larger coil/loop may not be injected easily and can be somewhat invasive than would be ideal in at least some instances. Also, the non-metal housing of at least some current device can result in an increased wall thickness to maintain hermetic sealing and structural stability may further increase the invasiveness of the device in at least some instances.

Therefore, a need exists for an injectable device that is less invasive and provides patient measurements and communication. Ideally, such improved devices will overcome at least some of the above limitations of the present methods and devices.

2. Description of the Background Art

The following U.S. Patent and Publications may be relevant to the present application: 2007/0150009; 2007/0118039; 2005/0080346; U.S. Pat. Nos. 7,295,879; and 6,658,300.

### BRIEF SUMMARY OF THE INVENTION

Embodiments of the present invention provide an injectable device that can be injected into a patient with decreased invasiveness so as overcome at least some of the above limitations. The implantable device comprises a first narrow profile configuration for injection so as to decrease invasiveness during injection through the skin of the patient, and a second expanded profile configuration so as to improve charging, communication and anchoring when the device is implanted in the patient.

In a first aspect, an injectable electronics device is provided. The device comprises a housing sized to fit within an injection tool lumen with one or more electrical components positioned within the housing and a self-expanding loop antenna coupled to at least one electrical component within the housing. The self-expanding loop antenna is expandable from a first compressed shape to a second expanded shape.

In another aspect, an injectable electronics device is provided. The device comprises an electronics package sized to fit within an injection tool lumen and a self-expanding wire loop coupled to the electronics package. The self-expanding wire loop is expandable from a first compressed shape to a second expanded shape.

In another embodiment, a method of implanting an injectable electronics device is provided. The method comprises providing an injection tool having a lumen and an injectable electronics device. The injectable electronics device includes an electronics package sized to fit within an injection tool lumen and a self-expanding wire loop coupled to the electronics package. The self-expanding wire loop is expandable from a first compressed shape to a second expanded shape. The method further comprises compressing the self-expanding wire loop, and loading the injectable electronics device within the injection tool lumen. A delivery end of the injection tool is positioned at a desired location of a patient, and the injectable electronics device is delivered from the injection tool lumen at the desired location.

In many embodiments, the compressed shape of the self-expanding loop fits within the injection tool lumen.

In many embodiments, the compressed shape may be an ellipse.

In many embodiments, the expanded shape is optimized for charging and/or communication with other electronic devices.

In many embodiments, the other electronic devices are located within a patient's body.

In many embodiments, the other electronic devices are located outside a patient's body.

In many embodiments, the self expanding loop antenna is configured such that the one or more loops extend at least partially around an area defined by the loop in the expanded shape, and the self expanding loop antenna is configured to expand such that the area is oriented toward a skin of the patient. This orientation of the area toward the skin of the patient can increase electromagnetic flux through the self expanding loop antenna.

In many embodiments, the expanded shape is planer, for example such that the loop extends substantially along a plane.

In many embodiments, the expanded shape is parallel to a patient's skin, for example substantially parallel to the skin of the patient.

In many embodiments, the self-expanding loop is constructed of a superelastic metal. The superelastic metal may comprise at least one of nitinol, stainless steel, MP35N or other metals that have been processed to provide elastic properties.

In many embodiments, the antenna is insulated with a material comprising at least one of ethylene tetrafluoroethylene (ETFE), polytetrafluoroethylene (PTFE), silicone or polyurethane.

In many embodiments, the self-expanding loop includes one or more loops in the expanded shape. The one or more loops may be in the same plane, or the one or more loops may be in multiple planes. For example, the one or more loops may extend substantially along the same plane or may extend substantially along multiple planes.

In many embodiments, the housing is made of metal, such as titanium.

In many embodiments, the self-expanding loop anchors the injectable device within the patient.

### BRIEF DESCRIPTION OF THE DRAWINGS

FIG. 1 shows an injectable electronics device having a communications and/or anchor loop, in accordance with embodiments;

FIG. 2 shows the injectable electronics device being loaded in a syringe-like injection tool used to deliver the device, in accordance with embodiments;

FIGS. 3A and 3B show an injection tool implanting the injectable electronics device within a patient's body;

FIG. 4 shows the injectable electronics device communicating with a pacing device, in accordance with embodiments;

FIG. 5 shows the injectable electronics device communicating with one or more recharging coils positioned on a mat the patient lays on, in accordance with embodiments;

FIG. 6A shows an injection tool having an alignment mark and an alignment structure configured to orient the injectable electronics device when injected into the patient in accordance with embodiments;

FIG. 6B shows a cross section of the injection tool as in FIG. 6A, in which the lumen of the injection tool has an alignment structure comprising an oval;

FIG. 7 shows the injection tool having one or more flanges to align the loop when inserted into the lumen, in accordance with embodiments;

FIG. 8A shows the injection tool comprising a recess and the injectable electronics device comprising a protrusion, in which the injectable electronics device is configured for placement in the injection tool; and

FIG. 8B shows the injectable electronics device positioned within the injection tool, in accordance with embodiments.

### DETAILED DESCRIPTION OF THE INVENTION

Embodiments of the present invention are directed to an injectable device having a deformable loop antenna for charging, communication and anchoring the device. The deformable loop antenna and methods of injection described herein can be used with many implantable or injectable medical devices, and can be especially helpful for those devices that use a loop for electromagnetic charging and communication. The expandable loop can also be used for device anchoring and stability. The embodiments described herein can be used with devices implanted and/or injected in many parts of the body and for many therapies, diagnoses, and additional treatments, for example as described in co-pending U.S. application Ser. No. 12/209430, entitled “INJECTABLE DEVICE FOR PHYSIOLOGICAL MONITORING” and co-pending U.S. application Ser. No. 12/209479, entitled “DELIVERY SYSTEM FOR INJECTABLE PHYSIOLOGICAL MONITORING SYSTEM”. Other delivery systems may include a catheter, an introducer, a needle, or any tube used for injection or delivery of an injectable device.

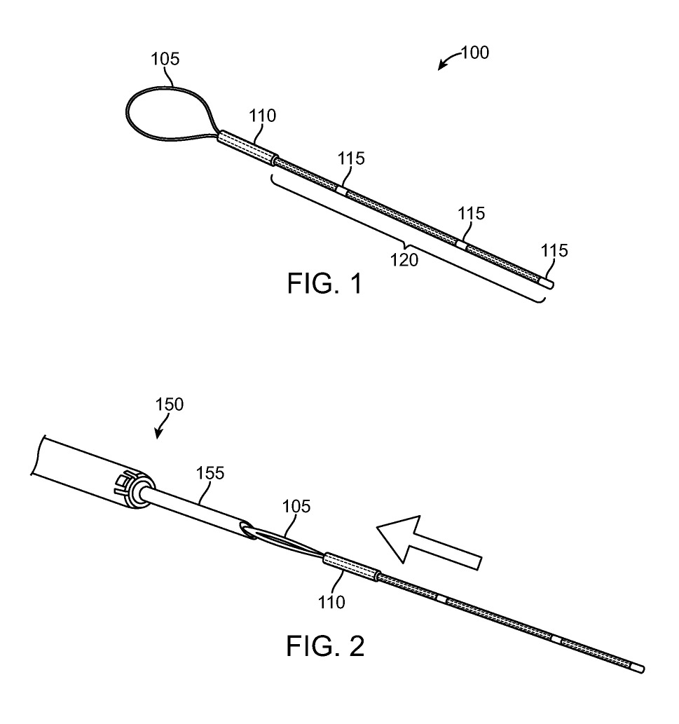

FIG. 1 shows embodiments of an injectable electronics device 100 having a communications and/or anchor loop 105, an electronics package 110 comprising electronics circuitry within a housing, and one or more sensors 115 positioned on a flexible body 120. The loop 105 may be used for communication and/or charging the electronics, and may also be used for anchoring the injectable electronics device 100 at a desired location within a body. The sensors are also coupled to the electronics.

The electronics package 110 has an outside diameter (OD) that can be slightly less than or equal to an inner diameter (ID) of a lumen of a delivery system. The OD of the electronics package 110 can be minimized by utilizing a metal material to reduce the wall thickness while maintaining structural stability and hermeticity. The electronics housing may be made of a variety of implantable materials with the primary option being titanium. Other bio-compatible metals may be used. The metal housing may also shield the electronics, such that electronics package does not interfere with communication from the antenna, which is outside electronics package.

The loop 105 is compressible or collapsible such that the loop 105 can be compressed to fit within the lumen of the delivery system. The loop 105 is also self-expanding, so as it is deployed from delivery system lumen, the loop 105 expands to create a large cross-sectional-area coil. The expanded coil of the loop can be planer. The expanded large cross-sectional area coil significantly reduces that amount of energy required to communicate and/or charge the device compared to a coil antenna within the electronics package that is limited to the size of the electronics package. The loop 105 may be constructed of a variety of metals: superelastic metals including Nitinol, stainless steel, MP35N or other metals that have been processed to provide elastic properties (i.e., can be compressed into a lumen without plastic deformation of the original loop shape). The loop 105 may also be insulated with a variety of polymers including ETFE, PTFE, silicone, or polyurethane.

The loop 105 shown in the FIG. 1 has one substantially planar loop or coil. For example, the substantially planar loop may extends substantially along the plane. In other embodiments, the loop may have multiple loops substantially in the same plane or multiple loops substantially in multiple planes, so that communication and or charging can occur more efficiently at varied angles relative to the device that is charging/being communicated with.

A loop antenna can be very directional, and may have a pickup pattern shaped like a figure eight, for example. The loop antenna can allow signals on opposite sides to be received, while off the sides of the loop antenna the signal can decrease or be nulled out. For this reason, it can be helpful to place the loop antenna in the proper orientation when it is injected. For communication with devices outside the body, the loop antenna can be oriented with the skin disposed over the antenna. For example, the loop antenna may comprise a substantially planar configuration that extends along a plane substantially parallel to the skin, such that the area of the loop is oriented toward the skin. The self expanding loop 105 can be configured such that the one or more loops extend at least partially around an area defined by the loop in the expanded shape. The self expanding loop antenna can be configured to expand such that the area is oriented toward a skin of the patient, for example when the package 110 is injected at a desired location in the patient with a desired orientation and position determined by an axis of the injection tool and a depth of the tip of the injection tool, respectively. This orientation of the area toward the skin of the patient can increase electromagnetic flux through the self expanding loop antenna. For communication with internal devices, the planer loop antenna axis and/or area can be pointed at the internal device.

FIG. 2 shows embodiments of the injectable electronics device 100 being loaded in a syringe-like injection tool 150 used to deliver the device. The injection tool includes a tip 155 having a lumen sized to receive the injectable electronics device 100. The injection tool 150 may also include a stylet or other wire or pusher to push the injectable electronics device 100 containing the loop 105 out of the lumen. The injection tool 150 may utilize a slider or ratcheted mechanism with a syringe or pistol grip.

Referring again to FIG. 2, the loop 105 is compressed or collapsed in size to fit the lumen. In the embodiments shown, the loop is pulled longitudinally, forming an ellipse shape sized to fit in the lumen. Once collapsed, the loop 105 is inserted into the lumen of the tip 155, followed by the rest of the injectable electronics device 100 including the electronics circuitry housing 110 and flexible body 120 with sensors 115. In other embodiments, the injectable electronics device 100 may be inserted into the lumen in the opposite direction, with the loop 105 going in last.

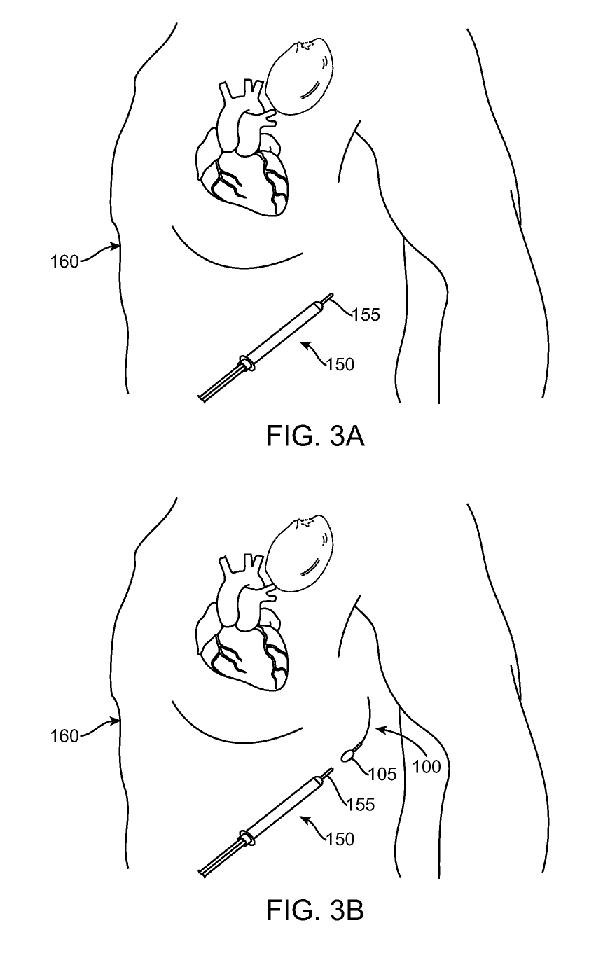

FIGS. 3A and 3B show an injection tool 150 implanting the injectable electronics device 100 within a patient's body 160. The injectable electronics device 100 may be implanted in any suitable area within the body 160, depending on the type of injectable electronics device 100. In the embodiment shown, the injectable electronics device 100 is implanted subcutaneously in the patient's side. The tip 155 is inserted into the body 160 at the desired location and the injection tool 150 dispenses the injectable electronics device 100. The injection tool may then be removed. After the injectable electronics device 100 is implanted, the loop 105 expands, preferably in the desired orientation for communication and/or charging. The loop 105 also anchors the injectable electronics device 100 at the desired location.

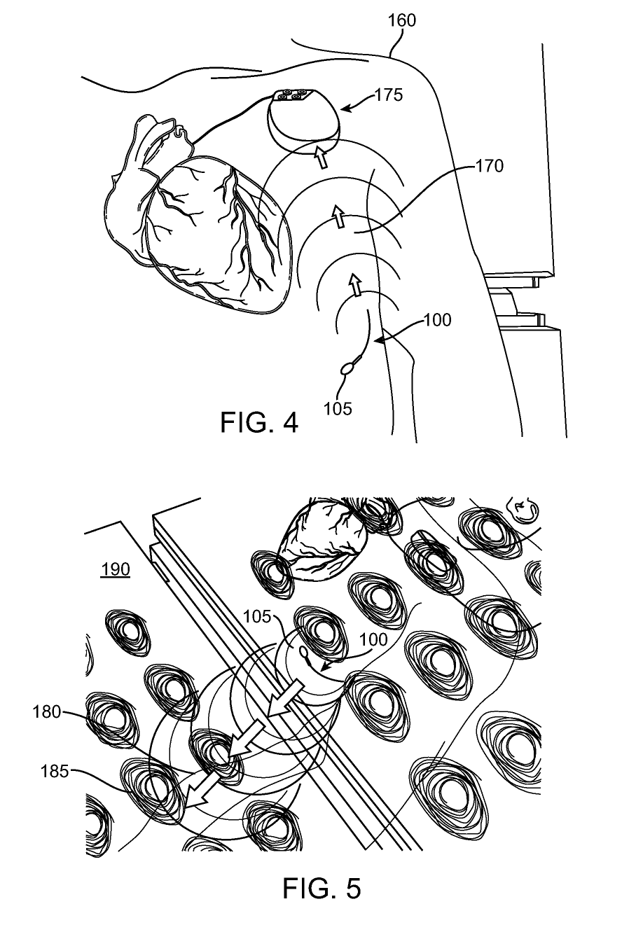

The loop 105 allows the injectable electronics device 100 to communicate with devices within the patient's body or external devices outside the body. FIG. 4 shows one embodiment of the injectable electronics device 100 communicating 170 with a pacing device 175. FIG. 5 shows one embodiment of the injectable electronics device 100 communicating 180 with one or more recharging coils 185 positioned on a mat 190 the patient lays on. Electromagnetic charging/communication can occur via inductive, RF, or by other electromagnetic transmission. Recharging of the sensors/battery and data transfer can occur while the patient is sleeping on the mat. The rechargeable batteries can also be transcutaneously charged with an external unit other than the mat.

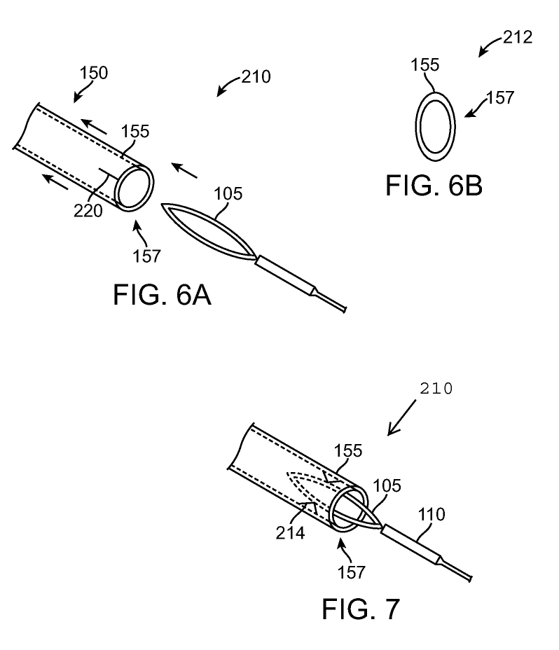

FIG. 6A shows the injection tool 150 having a structure 210 to align the loop 105 inserted into the lumen 157, and an alignment mark 220 that can be aligned with the patient, such that the device can be injected into the patient with a desired orientation. The structure 210 can be the cross section 212 of the lumen 157 as shown in FIG. 6B. The cross section 212 shown comprises an oval sized to receive and compress the loop 105, for example an oval comprising an ellipse. Further embodiments can include additional types of cross sections. This alignment of the loop 105 with the injection tool 150 can promote a more precise placement of the loop 105 when released.

Alignment may also be accomplished with the injection tool 150 having a mark 220 to orient the loop 105 for injection. The mark 220 may comprise a line drawn on the injection tool 150. Mark 220 may also comprise an indentation or other indicia for example.

Further embodiments may include a sliding mechanisms to align the loop 105 with the injection tool 150.

FIG. 7 shows the injection tool 150 having one or more flanges 214 to align the loop 105 when inserted into the lumen 157. When the loop 105 is received within the lumen 157, the flanges 214 engage the loop 105, the housing 110 or both, so as to align the loop with the injection tool. The flanges 214 also can disengage the loop 105 so as to release the injectable electronics device 100 at a desired orientation and position when aligned to the patient with mark 220. Engaging of the flanges 214 can be accomplished in various ways, such as automatic, electronic, or manual means. The structure 210 can be combined with the flanges 214 of FIG. 7. For example, the structure 210 can provide alignment to the loop 105 when received within the lumen 157. When the loop 105 is received, the flange 214 can engage the loop 105.

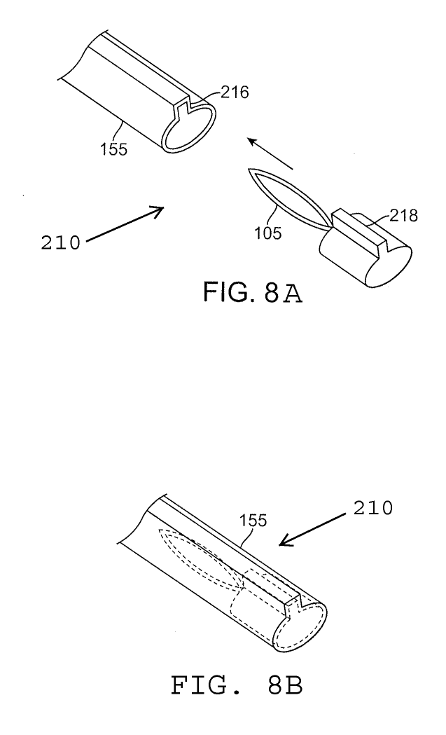

FIG. 8A shows the injection tool 150 having a recess 216 and the injectable electronics device 100 having a protrusion 218. The loop comprises an expanded shape configuration. When the injectable electronics device 100 is pulled longitudinally within the lumen 157, the protrusion 218 on the injectable electronics device 100 engages the recess 216. Once received within the lumen 157, the engaged protrusion 218 inhibits the electronics device 100 from internal rotation with respect to the injection tool 150, thus maintaining alignment. The protrusion 218 can be located on the lumen 157 and the recess 216 can be located on the electronics device 100.

FIG. 8B shows the injectable electronics device 100 positioned within the injection tool with the loop comprising a compressed shape configuration.

While the exemplary embodiments have been described in some detail, by way of example and for clarity of understanding, those of skill in the art will recognize that a variety of modifications, adaptations, and changes may be employed. Hence, the scope of the present invention should be limited solely by the appended claims.

What is claimed is:

1. An injectable electronics device comprising:
a housing sized to fit within an injection tool lumen;
one or more electrical components positioned within the housing; and
a self-expanding loop antenna coupled to at least one electrical component within the housing, the self-expanding loop antenna being expandable from a first compressed shape to a second expanded shape.

2. The device of claim 1, wherein the compressed shape fits within the injection tool lumen.

3. The device of claim 1, wherein the compressed shape comprises an ellipse.

4. The device of claim 1, wherein the housing has an outer diameter (OD) and the compressed shape is equal to or less than the OD of the housing.

5. The device of claim 1, wherein the expanded shape is optimized for charging and/or communication with other electronic devices.

6. The device of claim 5, wherein the other electronic devices are configured to be located within a patient's body.

7. The device of claim 5, wherein the other electronic devices are configured to be located outside a patient's body.

8. The device of claim 1, wherein the expanded shape is substantially planer.

9. The device of claim 1, wherein the expanded shape is substantially parallel to a patient's skin.

10. The device of claim 1, wherein the self-expanding loop antenna is constructed of a superelastic metal.

11. The device of claim 10, wherein the superelastic metal comprises at least one of nitinol, stainless steel, MP35N or other metals that have been processed to provide elastic properties.

12. The device of claim 1, wherein the antenna is insulated with a material comprising at least one of ethylene tetrafluoroethylene (ETFE), polytetrafluoroethylene (PTFE), silicone or polyurethane.

13. The device of claim 1, wherein the self-expanding loop antenna includes one or more loops in the expanded shape.

14. The device of claim 13, wherein the one or more loops extend along the same plane.

15. The device of claim 13, wherein the one or more loops extend along multiple planes.

16. The device of claim 13, wherein the self expanding loop antenna is configured such that the one or more loops extend at least partially around an area defined by the loop in the expanded shape and wherein the self expanding loop antenna is configured to expand such that the area is oriented toward a skin of the patient.

17. The device of claim 1, wherein the housing comprises metal.

18. The device of claim 17, wherein the housing comprises titanium.

19. The device of claim 1, wherein the housing is hermetically sealed.

20. The device of claim 1, wherein the self-expanding loop antenna is configured to anchor the injectable device within the patient.

21. The device of claim 1, wherein the injection tool lumen comprises a structure to align the self-expanding loop antenna with the injection tool lumen.

22. The device of claim 21, wherein the injection tool lumen comprises a cross section, wherein the cross section comprises the structure to align the self-expanding loop antenna.

23. The device of claim 22, wherein the cross section comprises at least one of an ellipse or an oval sized to receive the self-expanding loop antenna.

24. The device of claim 21, wherein the structure comprises at least one of a flange or a tab that engages at least one of the self-expanding loop antenna or the housing.

25. The device of claim 21, wherein the structure comprises at least one of a recess or a protrusion that engages at least one of the self-expanding loop antenna or the housing.

26. The device of claim 1, wherein the injection tool comprises a mark to orient the self-expanding loop antenna for injection.

27. The device of claim 26, wherein the mark comprises at least one of an indentation, a line, or indicia.

28. An injectable electronics device comprising:
an electronics package sized to fit within an injection tool lumen; and
a self-expanding wire loop coupled to the electronics package, the self-expanding wire loop being expandable from a first compressed shape to a second expanded shape.

29. The device of claim 28, wherein the self-expanding wire loop comprises an anchor.

30. The device of claim 28, wherein the self-expanding wire loop comprises a communication antenna coupled to at least one electrical component within the electronics package.

31. The device of claim 28, wherein the self-expanding wire loop comprises a combination communication antenna and anchor, the communication antenna being coupled to at least one electrical component within the electronics package.

32. The device of claim 28, wherein the self-expanding wire loop comprises an inductive coil loop.

33. The device of claim 28, wherein the self-expanding loop wire loop includes one or more loops in the expanded shape.

34. The device of claim 33, wherein the one or more loops extend substantially along the same plane.

35. The device of claim 33, wherein the one or more loops extend substantially along multiple planes.

36. The device of claim 28, wherein the expanded shape is substantially planer.

37. The device of claim 28, wherein the expanded shape is substantially parallel to a patient's skin.

38. A method of implanting an injectable electronics device comprising:
providing an injection tool having a lumen;
providing an injectable electronics device having:
an electronics package sized to fit within an injection tool lumen; and
a self-expanding wire loop coupled to the electronics package, the self-expanding wire loop being expandable from a first compressed shape to a second expanded shape;
compressing the self-expanding wire loop;
loading the injectable electronics device within the injection tool lumen;
positioning a delivery end of the injection tool at a desired location of a patient; and
delivering the injectable electronics device from the injection tool lumen at the desired location.

39. The method of claim 38, wherein the expanded shape is substantially planer.

40. The method of claim 38, wherein the expanded shape is substantially parallel to a patient's skin.

41. The method of claim 38, wherein the self-expanding loop wire loop includes one or more loops in the expanded shape.

42. The method of claim 41, wherein the one or more loops extend substantially along the same plane.

43. The method of claim 41, wherein the one or more loops extend substantially along multiple planes.

44. The method of claim 38, wherein the self-expanding wire loop comprises an anchor.

45. The method of claim 38, wherein the self-expanding wire loop comprises a communication antenna coupled to at least one electrical component within the electronics package.

46. The method of claim 38, wherein the self-expanding wire loop comprises a combination communication antenna and anchor, the communication antenna being coupled to at least one electrical component within the electronics package.
